(19) United States
(12) Patent Application Publication
(10) Pub. No.: US 2003/0224057 A1
(43) Pub. Date: Dec. 4, 2003

(54) Continuous method for preparing pharmaceutical granules

(76) Inventors: Stephane Martin-Letellier, Genas (FR); Jean-Claude Le Thiesse, Saint Etienne (FR)

Correspondence Address: BURNS DOANE SWECKER & MATHIS L L P, POST OFFICE BOX 1404, ALEXANDRIA, VA, 22313-1404, US

(21) Appl. No.: 10/182,527

(22) PCT Filed: Jan. 24, 2001
(86) PCT No.: PCT/FR01/00225

(30) Foreign Application Priority Data
Feb. 4, 2000 (FR) 00/01457

(51) Int. Cl. A61K 9/14; B29B 9/00

(52) U.S. Cl. 424/489; 264/9

(57) ABSTRACT

The present invention relates to a process for formulating one or more pharmaceutical active principles in the form of granules, characterized in that the various ingredients to be granulated are introduced continuously and this mixture is granulated using a device comprising a chamber and at least one rotary stirring arm, and in the presence of an effective amount of a binder solution, until the said granules are obtained.

The invention relates to the granulation of active principle(s) for pharmaceutical compositions.

Granulation is, in general, a technique which makes it possible to increase the particle size of a powder. More specifically, its aim is to convert pulverulent solids into more or less strong and more or less porous aggregates of variable size, which are referred to as granules. These granules have better flow and mechanical properties than a simple dry mixture of the ingredients, and granulation makes it possible to avoid the phenomena of demixing which may be observed during a dry-mixing operation.

Three main granulation routes exist: the wet route, the molten route and the dry route. Dry granulation is generally preferred for products liable to acquire, under stress, cohesion between particles, for example dry-compacting of a mixture of ingredients. Molten granulation is generally carried out for heat-stable products for which granules of low porosity are desired, for example by extruding an active principle suspended in a molten polymer. As regards wet granulation, this requires the addition of a solution to the mixture of ingredients, the purpose of which is to act as a binder and thus to contribute towards the aggregation of the particles with each other. This third route thus generally involves carrying out a consecutive drying step.

The present invention relates more particularly to granulation in a pharmaceutical medium via the so-called wet route.

Conventionally, the formulation of pharmaceutical active principles in the form of granules is carried out sequentially. In a first step, the mixture of the various active principles and combined excipients is prepared. Formulation of the mixture in the form of granules is carried out consecutively in a second step, which is thus carried out separately from the first step.

In point of fact, continuous operations are developed so as to optimise, in terms of cost, the conversion processes proposed to obtain a final product.

In this regard, the present invention relates to a process for formulating one or more pharmaceutical active materials in the form of granules, characterized in that the various ingredients to be granulated are introduced continuously and their mixture is granulated using a single device comprising a chamber and at least one rotary stirring arm, and in the presence of an effective amount of a binder solution, until the said granules are obtained.

For the purposes of the present invention, “continuous” means that there is no interruption of the process of conversion of the active material. In particular, the operations for the introduction of the various ingredients and for the granulation are performed continuously.

The process claimed thus has the advantage of dispensing with the usually batchwise operations of a granulation process, resulting in potential economic gains.

Similarly, according to a preferred embodiment of the invention, an operation of drying of the granules obtained is also carried out continuously, i.e. consecutively to the granulation step.

The process of the invention is preferably carried out using a mixer 10 represented in FIG. 1, of cylindrical shape in communication with a feed hopper 12 and comprising a discharge 14 for the granules obtained. It is preferably a twin-screw mixer functioning continuously.

More specifically, the mixer 10 consists of a cylindrical tank 16, generally made of stainless steel. The tank is surrounded by a jacket 18 containing a heat-exchange fluid to ensure temperature control during the granulation step.

In the tank 16 are arranged two counter-rotatory stirring shafts 20 equipped with twisted stirring blades 22. These shafts are arranged in parallel side by side along the axis of the tank and are driven by the same motor 24.

The mixer 10 also comprises points for injecting liquid, in particular binder solution, either at the base or at the top of the mixer.

Furthermore, the tank is provided, in its upstream section, with an inlet 26 for solid active materials, to which is connected the outlet of the hopper 12.

Ingredient(s) to be formulated in the mixing chamber are introduced via the feed hopper 12.

The binder solution is introduced separately into the chamber. It is introduced therein by means of one or more injection points, and concomitantly with that of the various solid ingredients.

The binder solution is generally introduced at ambient temperature, i.e. between 15° C. and 40° C. According to one embodiment of the process claimed, the binder solution is introduced at a temperature above ambient temperature and preferably between 40° C. and 90° C.

The granulation is carried out rapidly by stirring the combination of compounds in the chamber by means of the stirring shaft(s) and preferably at a temperature of between 20° C. and 150° C. and preferably from about 20° C. to 80° C.

Generally, the average granulation time is of the order of a few minutes.

To carry out the process of the invention in particular with apparatus as described above, it is recommended, in order to maintain optimum granulation conditions, to ensure a feed rate of solid active materials of between 50 kg/h and 250 kg/h and preferably from about 60 kg/h to 180 kg/h, and a spin speed of about from 100 rpm to 400 rpm.

At the mixing chamber outlet, the granules can be dried continuously, preferably in a fluidized bed so as to conserve a residual degree of humidity tailored to the subsequent use of the granules.

The granules may also, where appropriate, be calibrated by forced passage through a calibrating mesh. Generally, the particle size is then determined by screening.

The particle size of particles is commonly defined by a coefficient of variation (CV). This CV represents the particle size distribution in percentage values; the greater the CV, the greater the spread of the particle size distribution.

This CV is evaluated as follows:

$$CV = 100 \times \frac{{d}_{84} - {d}_{16}}{2 \cdot {d}_{50}}$$

The definitions are as follows:

the mean diameter, d₅₀, is such that 50% by weight of the particles have a diameter greater or less than the mean diameter,

d₁₆ is the diameter for which 16% by weight of the particles have a diameter less than this diameter,

d₈₄ is the diameter for which 84% by weight of the particles have a diameter less than this diameter.

After the calibration step, the coefficient of variation of the products of the invention obtained after the process claimed ranges between 30% and 100%, preferably between 40% and 90%. Their particle size generally ranges between 100 μm and 800 μm with a d₅₀, which represents a mean size of the particles, generally of between 300 μm and 500 μm.

It is seen that the state of granulation of the product, obtained after the process claimed, is particularly satisfactory. The granulation of the product is complete since the granules contain only a small amount of fines. They are therefore not too dusty.

Advantageously, granules of this type are particularly suitable for tabletting and are thus appropriate for the formation of tablets.

Needless to say, this tabletting operation falls within the competence of a person skilled in the art. The pharmaceutical evaluation of the tablets thus obtained in terms of quality of the cohesion, the friability and the disintegration time of the tablets is also satisfactory and is demonstrated in the examples given below.

The pharmaceutical active materials which may be formulated according to the process claimed may be highly water-soluble, such as acebutalol hydrochloride, or sparingly water-soluble, such as paracetamol.

Among the active materials which may be used in the process according to the present invention, mention may be made, in a non-limiting manner, of non-steroidal anti-rheumatism drugs and anti-inflammatory drugs (ketoprofen, ibuprofen, flurbiprofen, indomethacin, phenylbutazone, allopurinol, nabumetone, etc.), opiate or non-opiate analgesics (paracetamol, phenacetin, aspirin, etc.), antitussive drugs (codeine, codethyline, alimemazine, etc.), psychotropic drugs (trimipramine, amineptine, chlorpromazine, phenothiazine derivatives, diazepam, lorazepam, nitrazepam, meprobamate, zopiclone, and derivatives of the cyclopyrrolone family, etc.), steroids (hydrocortisone, cortisone, progesterone, testosterone, prednisolone, triamcinolone, dexamethazone, betamethazone, paramethazone, fluocinolone, beclomethazone, etc.), barbiturates (barbital, allobarbital, phenobarbital, pentobarbital, amobarbital, etc.), antimicrobial agents (pefloxacin, sparfloxacin, derivatives of the quinolone family, tetracyclines, synergistins, metronidazole, etc.), drugs intended for treating allergies, in particular anti-asthmatic, antispasmodic and antisecretory drugs (omeprazole), cerebral vasodilators (quinacainol, oxprenolol, propranolol, nicergoline, etc.), cerebral protectors, liver protectors, therapeutic agents for gastrointestinal purposes, contraceptive agents, oral vaccines, antihypertensive agents and cardiovascular or cardioprotective agents such as beta-blockers and nitro derivatives.

The amount of active material included in the pharmaceutical granules prepared according to the process of the present invention may vary within a wide range. It is more particularly between 0.001% and 98% by weight of the total composition, the remainder being made up of the combined excipients.

The process claimed is particularly advantageous for granulating paracetamol (acetyl-para-aminophenol). In this particular case, it is preferred to use a starting material, paracetamol, having an overall particle size ranging between 2 μm and 200 μm with a d₅₀ size of from 20 μm to 70 μm and the CV of which is from about 60% to 150%.

The pharmaceutical active principles may be formulated with excipients which make it possible to obtain the usual desired properties of granules. These excipients may be diluents, such as lactose, sucrose or calcium phosphates; cohesion agents, for instance hydrophilic polymers such as polyvinylpyrrolidone, cellulose, cellulose derivatives (hydroxypropylmethyl-cellulose, ethylcellulose), natural gums, modified natural gums or synthetic gums (gelatin, carob gums, guar gums, xanthan gums, alginates, carrageenates), native or pre-cooked starches, disintegrating agents, such as native starches, super-disintegrating agents such as sodium starch glycolate; flow agents, such as silica or talc; lubricants, such as stearic acid, magnesium stearate or calcium stearate; preserving agents, such as potassium sorbate, citric acid or ascorbic acid. This combination of components is generally introduced into the device with the active principles to be granulated. However, these excipients may be incorporated partially or totally into the binder solution.

As more particularly regards the binder solution, it is generally water. This binder solution may incorporate a material which, by its nature, promotes the aggregation of the active material particles to be formulated to form granules. Binders such as polyvinylpyrrolidone, cellulose, cellulose derivatives (hydroxypropylmethylcellulose, hydroxy-propylcellulose), natural gums, modified natural gums or synthetic gums (gelatin, carob gums, guar gums, xanthan gums, alginates, carrageenates) and native or pre-cooked starches are especially suitable for this type of function.

The binder solution is generally used in a proportion of from 40% to 100% by weight of active materials to be granulated. In point of fact, its amount is very variable and is associated in particular with the characteristics of the ingredients to be formulated (solubility, hygroscopicity, particle size distribution, rheology) and with the usual desired properties (mechanical properties, particle size distribution). The adjustment of this amount is especially within the competence of a person skilled in the art.

A subject of the present invention is also the use of a device comprising a cylindrical tank 16, generally made of stainless steel, surrounded by a jacket 18 containing a heat-exchange fluid to ensure temperature control of the mixture during granulation, and in which are arranged in parallel and side by side along the axis of the tank and driven by the same motor 24, two counter-rotating stirring shafts 20 equipped with twisted stirring blades 22, to granulate at least one pharmaceutical active material.

The examples and figure which follow are given as non-limiting illustrations of the present invention.

### FIGURE

FIG. 1: diagrammatic representation of the granulation plant.

### MATERIALS AND METHODS

The procedure is identical for all the tests carried out (residence time measurements, granulation tests, etc.).

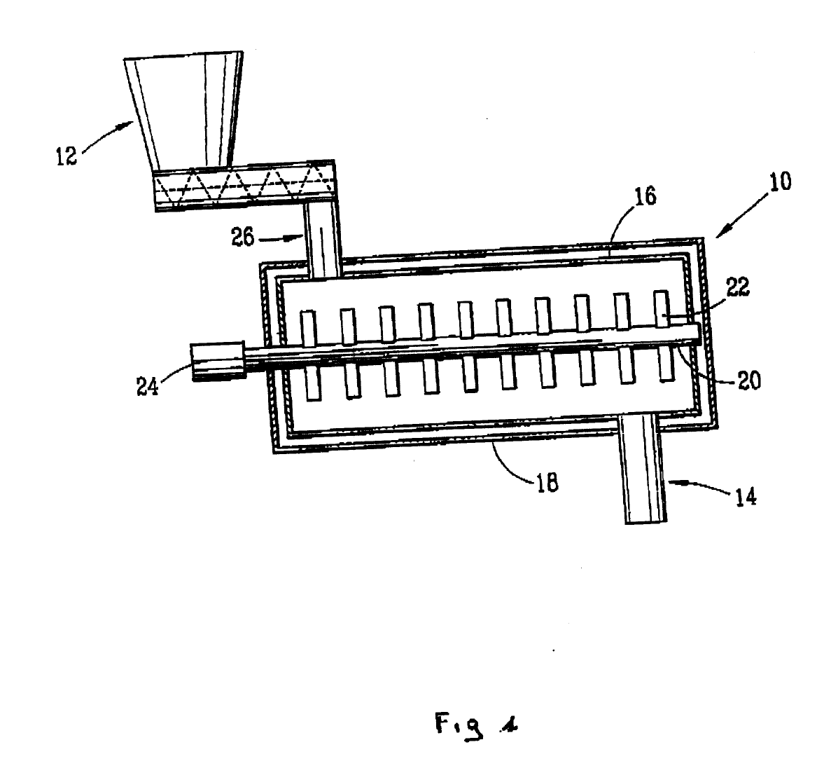

A device as represented in FIG. 1 is used.

The granulating machine is the twin-screw mixer operating continuously, described previously (Clextral 150 l preconditioner).

The motor for driving the mixing shafts has a power rating of 5.5 kW. The spin speed of the shafts is from 80 rpm to 420 rpm. A thermostatically maintained bath with circulation in its jacket is used.

A weight-loss metering hopper regulates a constant feed rate into the mixture. The outlet for the product from the mixer is at the bottom.

The tests used paracetamol or acetyl-para-aminophenol (Rhodapap Pulvérisé Dense NP, Rhodia) as active principle, the feed rate of which varied between 60 kg/h and 120 kg/h. The binder solution is composed of demineralized water and corn starch (Amidon de Maïs Extra-Blanc, Qualité Alimentaire [Extra-white corn starch, food grade], Roquette Frères) in a proportion of 20 kg of water per 3 kg of starch. This solution is cooked in a jacketed stirred tank at between 80° C. and 85° C. for at least 20 minutes in order to allow the starch to gelatinize, and is then injected into the mixer at a flow rate ranging between 20 kg/h and 60 kg/h.

### EXAMPLE 1

In this test, the feed rate of the paracetamol was set at 90 kg/h and that of the binder solution at 30 kg/h, for a spin speed of the mixer shafts of 290 rpm. Two samples of granules were taken from the mixer outlet and then dried to a residual moisture content of between 2.0% and 2.5%. They are finally calibrated using an 800 μm mesh. Table I below shows the results of the particle size distribution measured on the granules obtained after calibration.

TABLE I

| Sample | Date (mn) | d₁₀ (μm) | d₅₀ (μm) | d₉₀ (μm) | CV (%) |
|---|---|---|---|---|---|
| 1 | 26 | 145 | 391 | 690 | 55 |
| 2 | 42 | 145 | 391 | 696 | 55 |

The two samples were taken 26 min and 42 min, respectively, after the start of injection of the binder solution into the mixer. The particle size analysis by screening (Retsch AS 200 vibrating screen, 100 g sample, 1.5 mm vibrations, duration of 10 min) shows that the granules after the calibration step have a narrow size distribution illustrated by a low CV, and that the granulation is complete as illustrated by a high d₁₀ value, the active principle having at the start a d₅₀ value of between 2 μm and 80 μm. The granules obtained are thus very sparingly dusty.

### EXAMPLE 2

In this new test, the feed rate of the paracetamol was set at 90 kg/h and that of the binder solution was increased to reach 42 kg/h, for a spin speed of the mixing shafts maintained at 290 rpm. Three samples of granules were taken at the mixer outlet and were then dried to a residual moisture content of between 2.0% and 2.5%, and finally calibrated using an 800 μm mesh. Table II below presents the residual moisture content results of the granules leaving the mixer and the paracetamol titre of the granules obtained after calibration.

TABLE II

| | | Moisture | |
|---|---|---|---|
| Sample | Date (min) | content (%) | Titre (%) |
| 1 | 26 | 20.2 | 94.6 |
| 2 | 34 | 20.0 | 93.9 |
| 3 | 42 | 20.5 | 94.5 |

The residual moisture content is measured by weight loss on a Mettler PM460 thermobalance (sample of 3 g to 4 g, temperature of 105° C., duration of 15 min), while the paracetamol titre is obtained by fully dissolving the granules after drying and calibration, and comparing, against a reference sample, the absorption at 240 nm using a Perkin Elmer Lambda 20 UV spectrometer.

The results obtained show that the process is stable, with the samples of granules leaving the mixer at different dates having similar residual moisture contents. This stability is confirmed by the comparable paracetamol contents obtained on these same samples. Furthermore, homogeneity of the composition of the granules shows the advantage of this continuous granulation process for fixing a composition at the level of granules and avoiding the demixing phenomena which may be observed during a simple dry-mixing of the ingredients.

### EXAMPLE 3

One of the desired aims using a granulation process is to improve the usual properties of the starting active principles or their simple mixing with excipients. In the case of paracetamol, one of the essential properties which is desired is the compressability of the granules.

In this example, a disintegrating agent, namely corn starch (Amidon de Maìs Extra-Blanc, Qualité Alimentaire [Extra-white corn starch, food grade) Roquette Frères) and a lubricant, magnesium stearate (Magnésium Stéarate, Propharm) are added to the granules produced by means of this invention. These excipients are added to a dry-mixing machine (Retsch Bicone, stirring speed of 40 rpm, mixing time of 20 min). The mixture thus obtained is fed into a rotary press (Manesty 16-station Betapress, fed by a twin vane) in which are manufactured flat tablets, with a bevel, 12.5 mm in diameter.

The tablets are characterized by their geometrical properties (diameter, thickness), their mass and their mechanical properties (breaking strength, friability). These measurements are carried out using a Contestar machine and an Erweka TA 20 friability bench coupled with a Mettler PM 400 electronic balance. The cohesion of the tablets, expressed in MPa, is a magnitude calculated from the breaking strength and the geometrical data according to the following formula:

$$Cohesion = \frac{2 \times breaking strength}{π \times diameter \times thickness}$$

Tables III and IV below show the results of the measurements carried out on tablets of different formulation manufactured from the granules obtained according to the operating conditions described in Table 2.

TABLE III
Formulation: 1000.0 g granules of Example 2 + 61.5 g starch +
2.0 g magnesium stearate

| | | Friability | Cohesion |
|---|---|---|---|
| Compression (t) | Mass (mg) | (%) | (MPa) |
| 1.92 | 580.6 ± 3.8 | 0.26 | 0.87 |
| 2.51 | 583.6 ± 3.9 | 0.27 | 0.95 |
| 3.04 | 587.0 ± 5.3 | 0.23 | 0.97 |

TABLE IV
Formulation: 1000.0 g granules of Example 2 + 27.3 g
starch + 2.0 g magnesium stearate

| | | Friability | Cohesion |
|---|---|---|---|
| Compression (t) | Mass (mg) | (%) | (MPa) |
| 1.98 | 566.4 ± 3.2 | 0.29 | 0.73 |
| 2.50 | 566.1 ± 3.3 | 0.24 | 0.78 |
| 2.97 | 566.8 ± 2.9 | 0.51 | 0.65 |

These results were obtained using tablets manufactured at a pelletizing speed of 45,000 tablets/min, with the application of a precompression force of 0.5 t. The granulation substantially improves the flow properties of the simple mixture of the ingredients, as illustrated by the low standard deviation obtained for the mass of the tablets (standard deviation of less than 0.1%, generally of about 0.6%).

Furthermore, the tablets manufactured have satisfactory mechanical properties, as illustrated by the cohesion and friability data, given that the level of friability generally required by the European and American Pharmacopoeias, especially, is 1.0% maximum.

In general, the satisfactory progress of a step of pelletizing granulated active principles is very closely linked to the water content of these granules, since the water gives the granules a capacity for the plastic deformation required during the pelletizing step. The satisfactory behaviour of the granules produced by the present invention during the pelletizing step is thus very closely linked to the residual moisture content of the granules and to the satisfactory control of this parameter in the present continuous granulation process.

Moreover, the measurement of the paracetamol titre (according to the method described above, cf. § Example 2) on tablets obtained from the “1000.0 g granules Example 2+27.3 g starch+2.0 g magnesium stearate” formulation gives values equal to 88.6%±0.5%. This confirms the homogeneity of the granules obtained according to the continuous granulation process as described herein.

1. Process for formulating one or more pharmaceutical active principles in the form of granules, characterized in that the various ingredients to be granulated are introduced continuously and this mixture is granulated using a single device comprising a chamber and at least one rotary stirring arm, and in the presence of an effective amount of a binder solution, until the said granules are obtained.

2. Process according to claim 1, characterized in that an operation of drying of the said granules can be carried out in continuous mode consecutively to the granulation step.

3. Process according to claim 1 or 2, characterized in that the pharmaceutical materials are introduced into the chamber at a feed rate of between 50 kg/h and 250 kg/h.

4. Process according to one of the preceding claims, characterized in that the rotary stirring arm(s) is(are) subjected to a spin speed of about from 100 rpm to 400 rpm.

5. Process according to one of the preceding claims, characterized in that the chamber is maintained at a temperature of between 20° C. and 150° C. during the granulation operation.

6. Process according to one of the preceding claims, characterized in that the binder solution is introduced at ambient temperature or at a temperature above ambient temperature.

7. Process according to one of the preceding claims, characterized in that the binder solution is introduced separately into the chamber.

8. Process according to one of the preceding claims, characterized in that the coefficient of variation of the granules obtained after the calibration step ranges between 30% and 100% and preferably between 40% and 90%.

9. Process according to one of the preceding claims, characterized in that the mean size d₅₀ of the granules obtained after the calibration step is between 300 μm and 500 μm.

10. Process according to one of the preceding claims, characterized in that the active substances are chosen from non-steroidal anti-rheumatism drugs and anti-inflammatory drugs (ketoprofen, ibuprofen, flurbiprofen, indomethacin, phenylbutazone, allopurinol, nabumetone, etc.), opiate or non-opiate analgesics (paracetamol, phenacetin, aspirin, etc.), antitussive drugs (codeine, codethyline, alimemazine, etc.), psychotropic drugs (trimipramine, amineptine, chlorpromazine, phenothiazine derivatives, diazepam, lorazepam, nitrazepam, meprobamate, zopiclone, and derivatives of the cyclopyrrolone family, etc.), steroids (hydrocortisone, cortisone, progesterone, testosterone, prednisolone, triamcinolone, dexamethazone, betamethazone, paramethazone, fluocinolone, beclomethazone, etc.), barbiturates (barbital, allobarbital, phenobarbital, pentobarbital, amobarbital, etc.), antimicrobial agents (pefloxacin, sparfloxacin, derivatives of the quinolone family, tetracyclines, synergistins, metronidazole, etc.), drugs intended for treating allergies, in particular anti-asthmatic, antispasmodic and antisecretory drugs (omeprazole), cerebral vasodilators (quinacainol, oxprenolol, propranolol, nicergoline, etc.), cerebral protectors, liver protectors, therapeutic agents for gastrointestinal purposes, contraceptive agents, oral vaccines, antihypertensive agents and cardiovascular or cardioprotective agents such as beta-blockers and nitro derivatives.

11. Process according to one of the preceding claims, characterized in that the active material is paracetamol (acetyl-para-aminophenol).

12. Process according to claim 11, characterized in that the starting active material has an overall particle size of between 2 μm and 200 μm with a d₅₀ size of from 20 μm to 70 μm and a CV of about from 60% to 150%.

13. Process according to one of the preceding claims, characterized in that the device is in communication with a feed hopper 28 and consists of a cylindrical tank 16 surrounded by a jacket 18 containing a heat-exchange fluid to ensure temperature control of the mixture during granulation, and in which are arranged in parallel and side by side along the axis of the tank and driven by the same motor 24, two counter-rotating stirring shafts 20 equipped with twisted stirring blades 22.

14. Use of a device comprising a cylindrical tank 16, generally made of stainless steel, surrounded by a jacket 18 containing a heat-exchange fluid to ensure temperature control of the mixture during granulation, and in which are arranged in parallel and side by side along the axis of the tank and driven by the same motor 24, two counter-rotating stirring shafts 20 equipped with twisted stirring blades 22, to granulate at least one pharmaceutical active material.
